(19) United States
(12) Patent Application Publication
(10) Pub. No.: US 2019/0200722 A1
(43) Pub. Date: Jul. 4, 2019

(54) Portable Pedicure Station

(71) Applicant: Michael Solitt, Fort Lauderdale, FL (US)

(72) Inventor: Michael Solitt, Fort Lauderdale, FL (US)

(21) Appl. No.: 15/945,245

(22) Filed: Apr. 4, 2018

Related U.S. Application Data
(60) Provisional application No. 62/613,286, filed on Jan. 3, 2018.

(51) Int. Cl. A45D 29/00; A47C 16/02; A61B 17/54

(52) U.S. Cl. CPC A45D 29/00; A61B 17/54; A47C 16/025

(57) ABSTRACT

An apparatus for use in foot care or foot maintenance. The apparatus comprises a base plate, a back plate rotatably attached to the base plate, and a top plate rotatably attached to the back plate. The apparatus further comprises a kickstand pivotally attached to the top plate for engaging the base plate at a plurality of positions on the base plate. The apparatus may further comprise an accessory component attached to the back plate. A user adjusts the angle of the top plate with respect to the base plate by moving the kickstand along plurality of positions on the base plate. The user then places a foot on the top plate and uses the accessory component to assist in foot care or maintenance.

### CROSS-REFERENCE

This application claims priority from Provisional Patent Application Ser. No. 62/613,286 filed on Jan. 3, 2018.

### FIELD OF THE INVENTION

This invention pertains generally to an apparatus for use in foot care, and more particularly to a portable, freestanding and collapsible apparatus for conveniently enabling a user to safely and ergonomically provide foot and toe nail maintenance without the assistance of another.

### BACKGROUND

Foot care and toe nail grooming are lifelong necessities. Therapeutic and cosmetic treatments, such as foot massage, pedicures, and other foot grooming techniques are often performed by professionals in salons, spas, or podiatrist's offices. However, many individuals perform or wish to accomplish such treatments by themselves at home. Unfortunately, working on one's own feet can be difficult and uncomfortable for even the most limber individuals. Additionally, elderly, injured, and less flexible individuals find it difficult or impossible to attend to even the most basic of foot care needs, such as toe nail clipping or painting, at home. Foot care performed by one's self can even be dangerous as people bend and flex to attempt to clip, polish, or paint their toe nails. Another difficulty in home foot care is visibility. The awkward anatomical location of feet can make it difficult to adequately visualize toe nails and other foot areas that require maintenance. This can lead to injury as clippers, scissors, and other sharp instruments are typically used to maintain toe nails.

Consequently, there exists a long felt need in the art for an apparatus for allowing a user to care for one's own foot and toe maintenance needs at home that is safe and easy to use. The present invention effectively allows a user to comfortably reach and care for one's own feet. The invention is especially well suited for elderly individuals, or for anyone who has difficulty bending and stretching. The invention provides safe and easy access to feet for pedicure and other cosmetic treatments, as well as therapeutic treatments such as massage or skin care. The improved foot care apparatus is portable, freestanding, adjustable, and provides improved visibility and access to feet. The apparatus also folds up for easy transport and storage. Additionally, the invention improves visibility of the work area allowing for more accurate and safer foot care.

### SUMMARY

The following presents a simplified summary in order to provide a basic understanding of some aspects of the disclosed invention. This summary is not an extensive overview, and it is not intended to identify key/critical elements or to delineate the scope thereof. Its sole purpose is to present some concepts in a simplified form as a prelude to the more detailed description that is presented later.

The subject matter disclosed and claimed herein, in one aspect thereof, comprises a foot care apparatus for providing easy and improved access to a user's feet. The apparatus comprises a base plate assembly, a back plate assembly pivotally attached to the base plate assembly, a top plate assembly for engaging a foot and pivotally attached to the back plate assembly, and a kickstand for positioning and repositioning the top plate assembly with respect to the base plate assembly. The kickstand is preferably pivotally attached to the top plate assembly, and is adjustably positional along the base plate assembly.

Furthermore, the base plate assembly comprises a plurality of positioning slots for engaging the kickstand to change the orientation of the top plate assembly with respect to the base plate assembly. The foot care apparatus further comprises a lower hinge element pivotally connecting the base plate assembly and the back plate assembly, and an upper hinge element pivotally connecting the back plate assembly to the top plate assembly. The foot care apparatus is collapsible as the kickstand folds into the top plate assembly, the top plate assembly folds into the back plate assembly, and the back plate assembly folds into the base plate assembly.

The foot care apparatus further comprises an accessory component attachable to the back plate assembly. The accessory component may comprise one or more of the following: an illumination element, a focusing element, and a magnification element. The accessory component is pivotally attached to the back plate assembly and is positional with respect to the top plate assembly so as to improve a user's visibility of a foot when placed on the top plate assembly.

To the accomplishment of the foregoing and related ends, certain illustrative aspects are described herein in connection with the following description and the annexed drawings. These aspects are indicative of the various ways in which the principles disclosed herein can be practiced and all aspects and equivalents thereof are intended to be within the scope of the claimed subject matter. Other advantages and novel features will become apparent from the following detailed description when considered in conjunction with the drawings.

### BRIEF DESCRIPTION OF THE DRAWINGS

FIG. 1 illustrates a perspective view of a foot care apparatus in accordance with the disclosed architecture.

FIG. 2 illustrates a rear perspective view of the foot care apparatus in accordance with the disclosed architecture.

FIG. 3 illustrates a front view of the foot care apparatus in accordance with the disclosed architecture.

FIG. 4 illustrates a rear view of the foot care apparatus in accordance with the disclosed architecture.

FIG. 5 illustrates a side view of the foot care apparatus in accordance with the disclosed architecture.

FIG. 6 illustrates a side view of the foot care apparatus in accordance with the disclosed architecture.

FIG. 7 illustrates an overhead view of the foot care apparatus in accordance with the disclosed architecture.

FIG. 8 illustrates an underneath view of the foot care apparatus in accordance with the disclosed architecture.

FIG. 9 illustrates an exploded schematic view of the foot care apparatus in accordance with the disclosed architecture.

FIG. 10 illustrates a top view of the foot care apparatus collapsed in accordance with the disclosed architecture.

FIG. 11 illustrates a bottom view of the foot care apparatus collapsed in accordance with the disclosed architecture.

FIG. 12 illustrates a perspective view of the foot care apparatus collapsed in accordance with the disclosed architecture.

FIGS. 13A-E illustrates a sequencing view of the foot care apparatus moving between a deployed position and a collapsed position in accordance with the disclosed architecture.

### DETAILED DESCRIPTION

Reference is now made to the drawings, wherein like reference numerals are used to refer to like elements throughout. In the following description, for purposes of explanation, numerous specific details are set forth in order to provide a thorough understanding thereof. It may be evident, however, that the novel embodiments can be practiced without these specific details. In other instances, well known structures and devices are shown in block diagram form in order to facilitate a description thereof. The intention is to cover all modifications, equivalents, and alternatives falling within the spirit and scope of the claimed subject matter. The invention relates generally to an apparatus for improving ones access to one's feet.

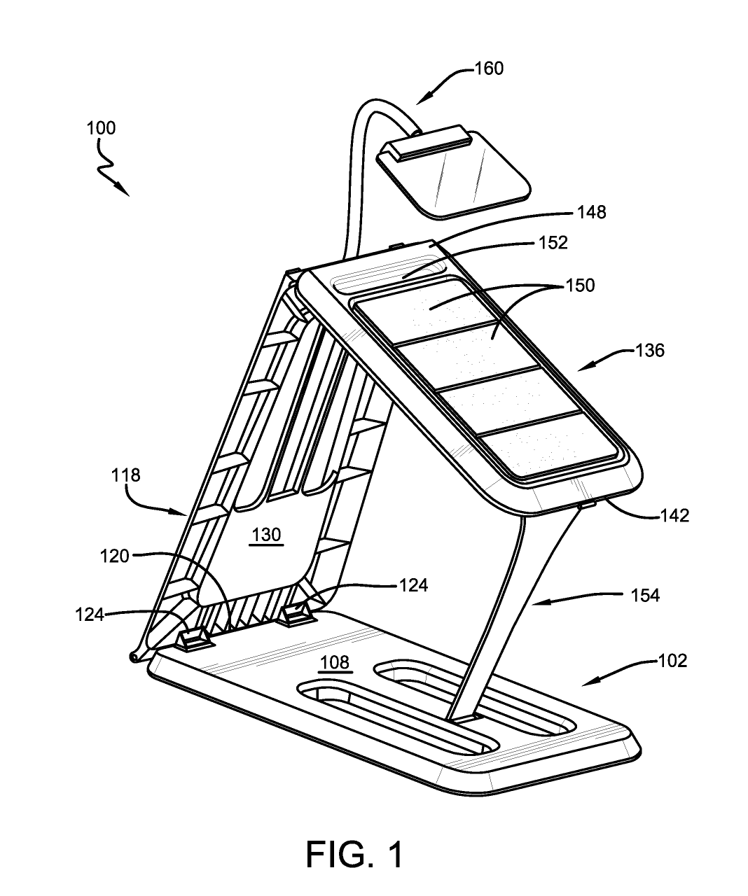

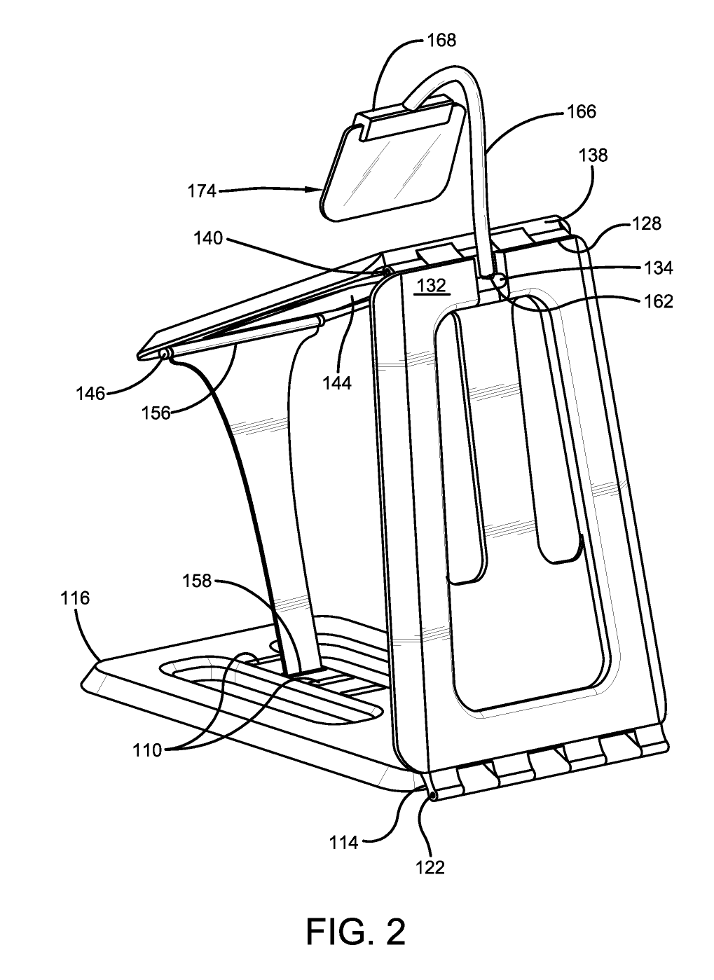

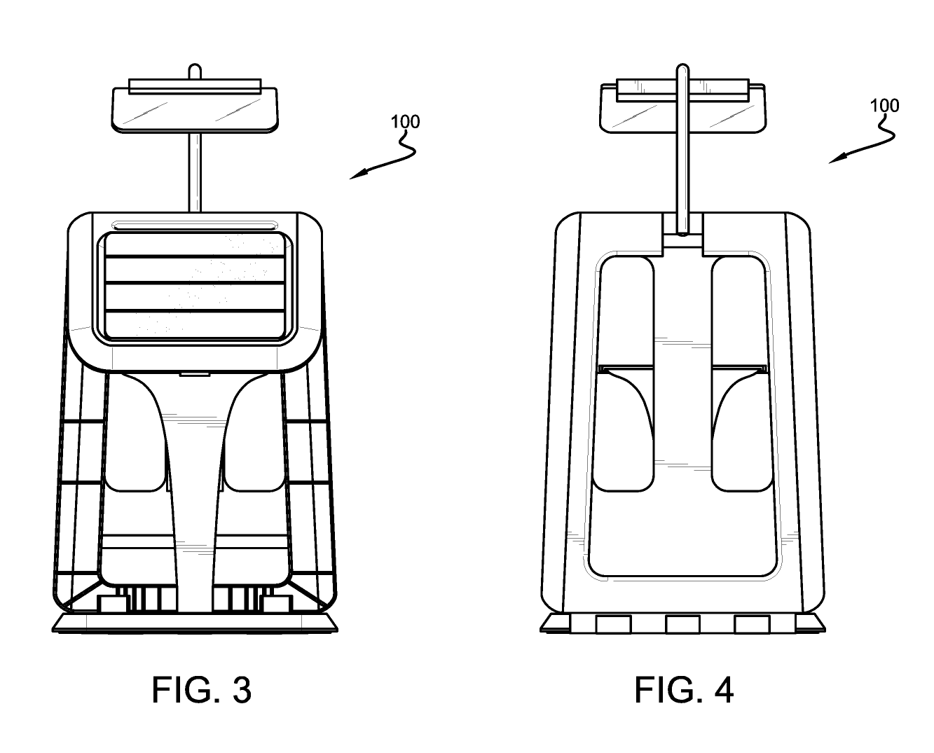

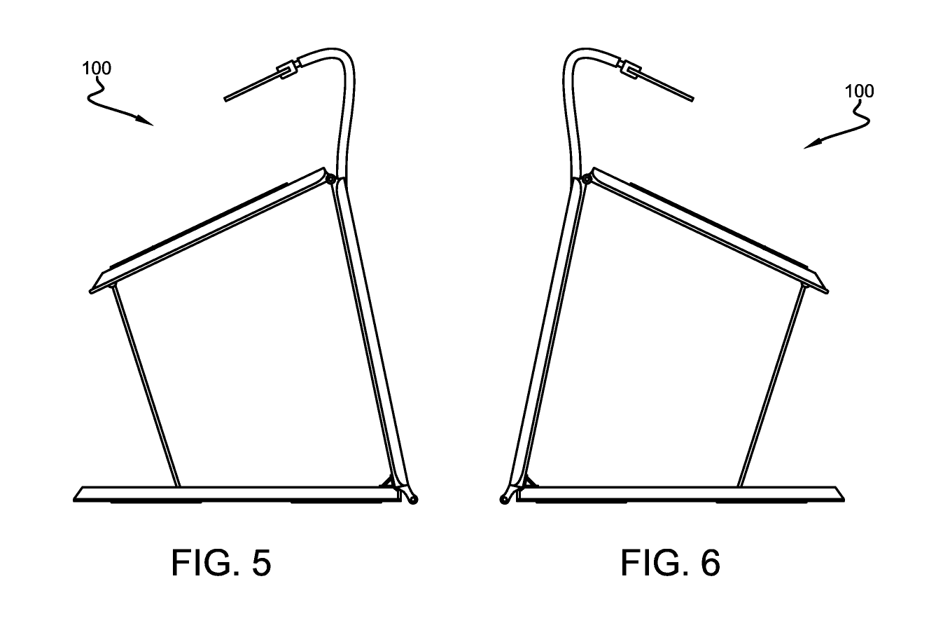

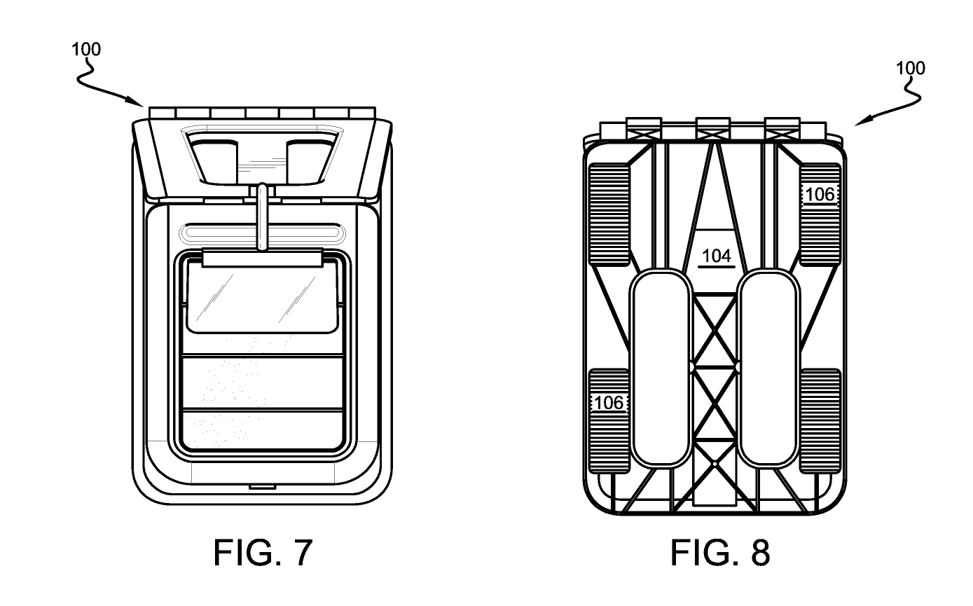

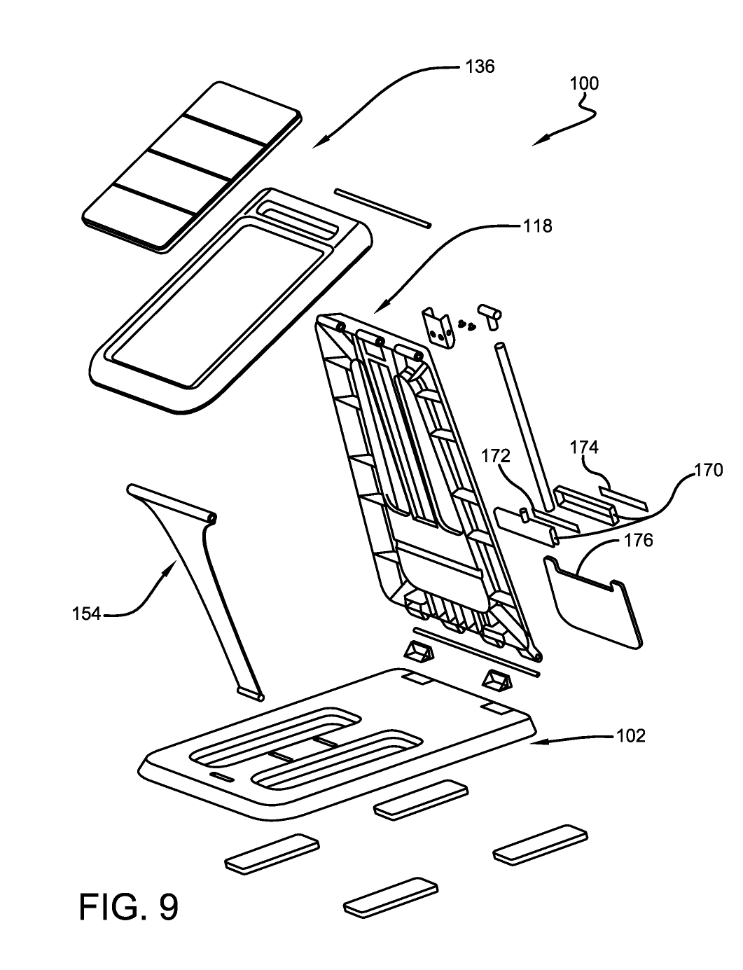

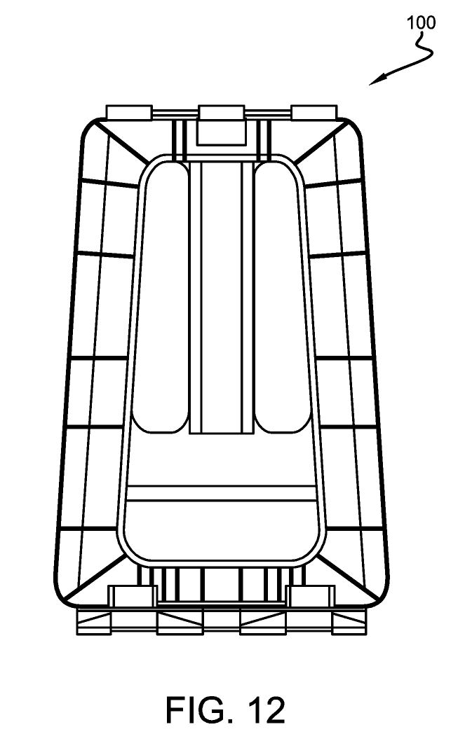

FIGS. 1-13 illustrate a foot care apparatus 100. The foot care apparatus 100 is designed to assist individuals with foot care or foot maintenance. The invention is especially beneficial in assisting the elderly or infirm to safely and comfortably clip one's own toe nails and perform other routine foot and toe maintenance, such as toe nail filing and painting. The invention is ergonomically designed to prevent straining or spraining to reach feet and toes, is adjustable in height, and is collapsible for portability and easy storage. Additionally, the invention is easy to deploy as a free standing portable pedicure station not enclosed in a box or external housing and does not require significant maintenance.

As illustrated in FIGS. 1-8, the foot care apparatus 100 comprises a base plate assembly 102, a back plate 118, a top plate 136, and a kickstand 154. The base plate assembly 102 is typically rectangular in configuration, although it can be any shape as desired as well. The base plate assembly 102 comprises a bottom side 104 and a top side 108. The base plate assembly 102 may further comprise a plurality of non-skid elements 106 fixable to the bottom side 104. The plurality of non-skid elements 106 may be feet constructed from a material capable of frictionally engaging a surface upon which the foot care apparatus 100 is placed. As a foot is placed on the top plate assembly 136, the downward pressure of the foot allows the plurality of non-skid elements 106 to engage the floor or other surface to prevent the foot care apparatus 100 from slipping or sliding along the floor or other surface. The plurality of non-skid elements 106 may alternatively have a rake-like texture for engaging a carpet surface to hold the foot care apparatus 100 in place, while the same is in use. The plurality of non-skid elements 106 may be attachable to the bottom side 108 of the base plate assembly 102 by adhesive, glue, fastener, or the like.

The base plate assembly 102 further comprises a plurality of positioning slots 110 cut, formed, etched, or molded into the top surface 108. The plurality of slots 110 may also be through holes as desired. The plurality of positioning slots 110 is configured to engage the kickstand 154 as described infra. The base plate assembly 102 further comprises a back edge 114 and a front edge 116. The plurality of positioning slots 110 is aligned from the front edge 116 inward in the direction of the back edge 114.

The back plate assembly 118 is typically trapezoidal in configuration, although it can be any shape as desired as well. The back plate assembly 118 comprises base plate engaging end 120, a top plate engaging end 128, an inner surface 130, and an outer surface 132. The foot care apparatus 100 further comprises a lower hinge element 122. The lower hinge element 122 is typically a cylindrical rod, pin, or the like that pivotally connects the base plate assembly 102 at the back edge 114 to the back plate assembly 118 at the base plate engaging end 120. This allows the back plate assembly 118 to be repositioned angularly with respect to the base plate assembly 102. The lower hinge element 122 allows for approximately 300 degree movement.

The foot care apparatus 100 further comprises a plurality of locking elements 124. The plurality of locking elements 124 may be wedges, or similar shaped components, positioned on the top side 108 of the base plate assembly 102 adjacent to the back edge 114, and along the inner surface 130 of the back plate assembly 118 at the base plate engaging end 120. The plurality of locking elements 124 are designed to temporarily hold the base plate assembly 102 at a desired angle with respect to the back plate assembly 118 when the foot care apparatus 100 is in use. When employed, the plurality of locking elements 124 temporarily fixes the orientation of the back plate assembly 118 with respect to the base plate assembly 102.

The top plate assembly 136 is typically rectangular in configuration, although it can be any shape as desired as well. The top plate assembly 136 is designed to engage a foot and comprises a bottom side 144, a top side 148, a back plate engaging end 138, and a kickstand engaging end 142. The top plate assembly 136 further comprises a kickstand mount 146 located on the bottom side 144 for engaging the kickstand 154 as described infra. The top plate assembly 136 may further comprise a foot engaging element 150 and a well 152 both positioned on the top side 148. The foot engaging element 150 may be a pad or otherwise cushioned, molded, padded, or arched area for receiving a foot. Alternatively, the area of the top plate assembly 136 adjacent to the foot engaging element 150 may be arched on either side to accommodate both left and right feet. The well 152 may be oval or rectangular in shape, and extends downward or is dug, molded, or formed into the top side 148. The well 152 is configured to receive and retain toe nail clippings for later disposal.

The foot care apparatus 100 further comprises an upper hinge element 140. The upper hinge element 140 is typically a cylindrical rod, pin, or the like similar to the lower hinge element 122 that pivotally connects the back plate assembly 118 at the top plate engaging end 128 to the top plate assembly 136 at the back plate engaging end 138. This allows the top plate assembly 136 to change position with respect to the back plate assembly 118. The upper hinge element 140 allows for approximately 300 degree movement.

The kickstand 154 comprises a top plate engaging portion 156 and a base plate engaging portion 158. The kickstand 154 allows the user to position or otherwise change the orientation of the top plate assembly 136 with respect to the base plate assembly 102. The top plate engaging portion 156 is movably or pivotally attached to the kickstand mount 146 on the bottom side 144 of the top plate assembly 136. The base plate engaging portion 158 is configured to engage the plurality of positioning slots 110 of the base plate assembly 102. As the base plate engaging portion 158 moves from slot to slot, the angle of the top plate 136 with respect to the base plate assembly 102 changes. This allows the user to select the most comfortable angle for their body. Additionally, the steeper the angle, the more friction is applied to the plurality of non-skid elements 106 keeping the foot care apparatus 100 stable when engaged by a foot.

The foot care apparatus 100 further comprises a mounting plate 134 attachable to the outer surface 132 of the back plate assembly 118, and an accessory component 160 pivotally attachable to the back plate assembly 118 via the mounting plate 134. The accessory component 160 may comprise a mounting element 162, a neck 166, and a housing 168. The mounting element 162 may be a gooseneck or T-type mount connectable to the mounting plate 134 with a rivet or similar connector. The neck 166 may be a goose neck or similar flexible member capable of extending over the top side 148 of the top plate assembly 136. The housing 168 is attached to the neck 166 opposite of the mounting element 162. The housing 168 may comprise a pair of holding elements 170 for engaging and retaining additional components as described infra.

The accessory component 160 may further comprise an illumination element 172. The illumination element 172 is typically a small battery powered light, such as an LED, which is attachable to the housing 168. The accessory component 160 may also further comprise a focusing element 174 attachable to the housing 168. The focusing element 174 may be a lens for directing light from the illumination element 172. The accessory component 160 may also further comprise a magnifying element 176 attachable to the housing 168. The magnifying element 176 is typically a magnifying glass to improve visibility for the user. The magnifying element 176 is extendable approximately horizontally over the top plate. Ample magnification may range from 1×-10× or greater as desired. Additionally the illumination element 172 may be arranged in position with the magnifying element 176 to reduce glare from instruments or tools such as nail clippers. The illumination element 172, the focusing element 174, and the magnifying element 176 may all be engaged and retained by the pair of holding elements 170 of the housing 168.

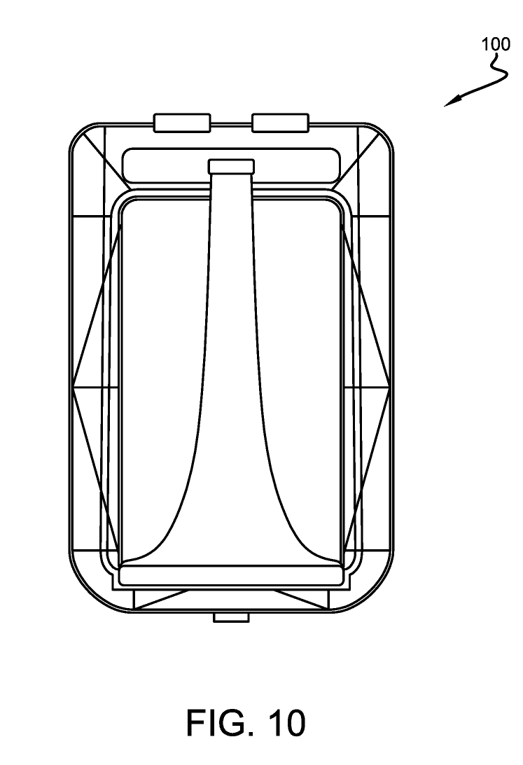

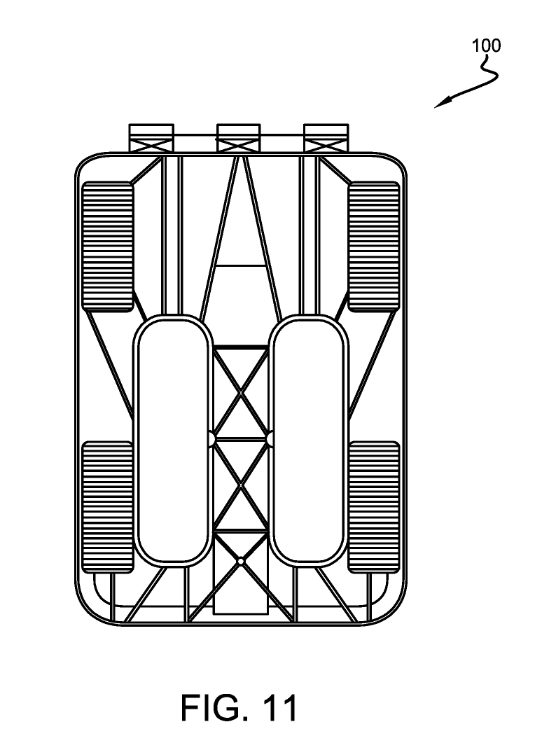

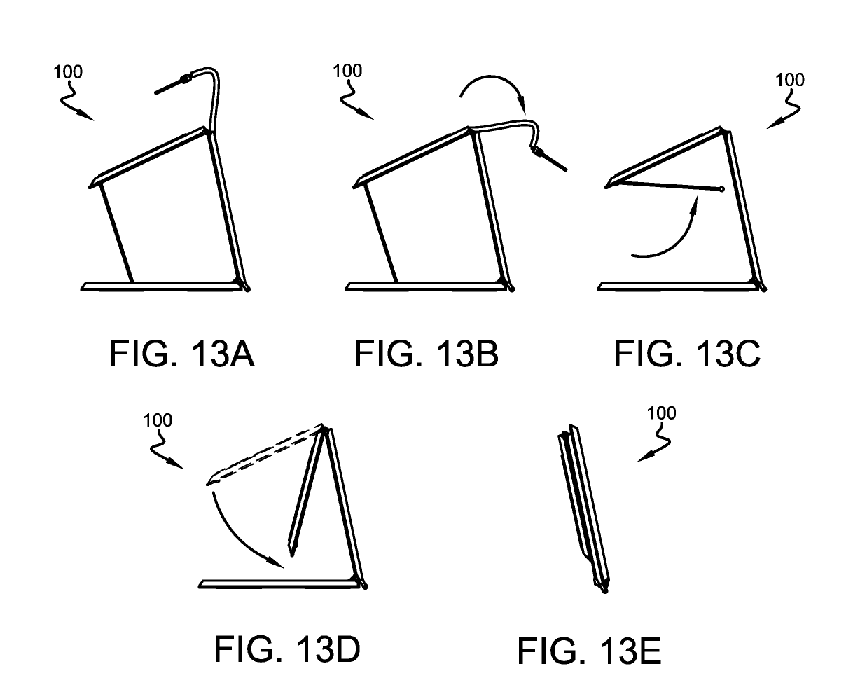

The foot care apparatus 100 is adjustable in height and angle to best suit a variety of users. This allows accommodation of users who have difficulty bending at the waist or lifting their legs. Typically a user may sit in a chair and place a foot on the adjusted top plate assembly 136. The magnification and lighting from the accessory component 160 reduces eye strain and guessing where to clip on small toe nails. The foot care apparatus 100 is also completely collapsible for storage as illustrated in FIGS. 10-13A-E. To collapse the foot care apparatus 100 from a deployed position as illustrated in FIG. 13A, the accessory component folds into the back plate assembly as illustrated in FIG. 13B. Then the base plate engaging portion 158 of the kickstand 154 is removed from one of the positioning slots 110 in the base plate assembly 102 and folds in to the top plate assembly 136 as illustrated in FIG. 13C. The top plate assembly 136 then folds into the back plate assembly 118, as illustrated in FIG. 13D. And, as illustrated in FIG. 13E, the base plate assembly 102 folds over the top plate assembly 136 into the back plate assembly 118 so that the foot care apparatus 100 is substantially flat when in a stored configuration.

Notwithstanding the forgoing, other variations are within the spirit of the present invention. Thus, while the invention is susceptible to various modifications and alternative constructions, a certain illustrated embodiment thereof is shown in the drawings and has been described above in detail. It should be understood, however, that there is no intention to limit the invention to the specific form or forms disclosed, but on the contrary, the intention is to cover all modifications, alternative constructions, and equivalents falling within the spirit and scope of the invention, as defined in the appended claims.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. The term “connected” is to be construed as partly or wholly contained within, attached to, or joined together, even if there is something intervening. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate embodiments of the invention and does not pose a limitation on the scope of the invention unless otherwise claimed. No language in the specification should be construed as indicating any non-claimed element as essential to the practice of the invention.

Preferred embodiments of this invention are described herein. Variations of those preferred embodiments may become apparent to those of ordinary skill in the art upon reading the foregoing description. The inventor expects skilled artisans to employ such variations as appropriate, and the inventor intends for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

What is claimed is:

1. A foot care apparatus comprising:
a base plate assembly;
a back plate assembly attached to the base plate assembly;
a top plate assembly attached to the back plate assembly; and
a kickstand for positioning the top plate assembly with respect to the base plate assembly.

2. The foot care apparatus of claim 1, wherein the base plate assembly comprises a plurality of non-skid elements.

3. The foot care apparatus of claim 1, wherein the base plate assembly comprises a plurality of positioning slots for engaging the kickstand.

4. The foot care apparatus of claim 1, wherein the top plate assembly comprises a foot engaging element.

5. The foot care apparatus of claim 1, wherein the top plate assembly is repositionable.

6. The foot care apparatus of claim 1, wherein the foot care apparatus is collapsible.

7. A free standing foot care apparatus comprising:
a base plate assembly;
a back plate assembly attached to the base plate assembly;
a top plate assembly attached to the back plate assembly;
a kickstand for positioning the top plate assembly with respect to the base plate assembly; and
an accessory component attached to the back plate assembly.

8. The foot care apparatus of claim 7, wherein the accessory element comprises an illuminating element.

9. The foot care apparatus of claim 7, wherein the accessory element comprises a focusing element.

10. The foot care apparatus of claim 7, wherein the accessory element comprises a magnifying element.

11. The foot care apparatus of claim 7, wherein the top plate assembly comprises a well.

12. The foot care apparatus of claim 7 further comprising an upper hinge element pivotally connecting the top plate assembly to the back plate assembly.

13. The foot care apparatus of claim 7, wherein the foot care apparatus is collapsible.

14. A collapsible foot care apparatus comprising:
a base plate assembly;
a back plate assembly pivotally attached to the base plate assembly;
a top plate assembly for engaging a foot pivotally attached to the back plate assembly; and
a kickstand pivotally attached to the top plate assembly for changing an orientation of the top plate assembly with respect to the base plate assembly.

15. The foot care apparatus of claim 14 further comprising an accessory component pivotally attached to the back plate assembly.

16. The foot care apparatus of claim 15, wherein the accessory component comprises at least one of an illuminating element, a focusing element, or a magnifying element.

17. The foot care apparatus of claim 14, further comprising a lower hinge element pivotally connecting the back plate assembly to the base plate assembly.

18. The foot care apparatus of claim 17, further comprising a plurality of locking elements for fixing an orientation of the back plate assembly with respect to the base plate assembly.

19. The foot care apparatus of claim 14, wherein the base plate assembly comprises a plurality of positioning slots for engaging the kickstand.

20. The foot care apparatus of claim 14, wherein the base plate assembly, the back plate assembly, the top plate assembly, and the kickstand fold up substantially flat.
